(19) United States
(12) Patent Application Publication
(10) Pub. No.: US 2026/0294430 A1
(43) Pub. Date: Oct. 1, 2026

(54) A SURGICAL STAPLER

(71) Applicant: Gosai Gaurangkumar ASHWINGIRI, GANDHINAGAR, GUJARAT (IN)

(72) Inventor: Gosai Gaurangkumar ASHWINGIRI, GANDHINAGAR, GUJARAT (IN)

(21) Appl. No.: 19/480,719

(22) PCT Filed: May 3, 2024
(86) PCT No.: PCT/IB2024/054332

§ 371 Date: Oct. 31, 2025

(30) Foreign Application Priority Data
May 4, 2023 (IN) 202321031768

(51) Int. Cl. A61B 17/068; A61B 17/00

(52) U.S. Cl. CPC A61B 17/0684; A61B 2017/00367

(57) ABSTRACT

A surgical stapler is disclosed having a stapler body with one end provisioned with a receptacle to receive an affected area of a specimen. A circular base is disposed proximal to the receptacle and defines a curvilinear path structured to guide one staple of a plurality of staples towards an outlet end defined in the receptacle. A drive mechanism is structured to displace the one staple of the plurality of staples. The drive mechanism comprises a conveying member being configured to be displaced between a first position and a second position relative to the curvilinear path. An actuator operatively coupled to the conveying member. A deforming module disposed proximal to an inlet end of the receptacle and structured to deform a portion of the one staple. The applied staple resides partially within the tissue such that only the bridge and distal arm portions remain external to the skin surface.

### TECHNICAL FIELD

Present disclosure relates to the field of medical devices. Particularly, but not exclusively, embodiments of the present disclosure relate to surgical stapler.

### BACKGROUND OF THE DISCLOSURE

Generally, wounds and surgical incisions on tissue of a specimen must be closed for healing. Various techniques have been developed to close the wound or the surgical incisions such as, for example, suturing, applying clips to blood vessels, and using surgical fasteners and the like. Hereinafter referred to as would closure, is usually done by sutures, staplers, adhesives, or strips as described above. Closure with sutures is generally time-consuming, tedious, and not easy to perform for certain closure techniques. Another disadvantage of using a suture is possible strangulation and necrosis of the tissue due to high pressure exerted by relatively small surface area of the suture and over-tightening of the suture in order to tie the suture snugly. Sutures are of two types, a non-biodegradable and bio-degradable suture. While use of biodegradable sutures include exceedingly early loss of tensile strength, causes inflammation by the degradation products of the suture, and subsequent granulomas in the tissue, the non-bio-degradable sutures need to be removed after some days and if kept for longer periods may lead to scarring of the tissues. With myriad drawbacks posed by the sutures, surgical staplers are developed to close the wounds.

Surgical staplers are medical devices that may be used in place of sutures. The surgical staplers can close large wounds or incisions more quickly and may be less painful than stitches for patients. They can also be used to close wounds in areas where the skin is tight against bone, in operations to remove organs, or to reconnect parts of internal organs. Surgical staplers are frequently used to close incisions in the abdomen and uterus during caesarean deliveries, or C-sections.

Surgical staplers have recently started to be utilized in the healthcare sector including both human medicine and veterinary practices, for the purpose of suturing wounds and incisions. The surgical skin stapling is desirable in that it requires less skill of the health care worker/clinician than conventional suture ligature. In addition, the removal of staples is faster and easier than suture ligature. As in most fields, simplicity in wound and incision closure decreasing the possibility of an error is an accomplishment of particular importance in the healthcare field. The surgical stapling also provides a better post-surgery aesthetic appearance than conventional suture ligature and needle wound closure. Further, postoperative infection is reduced in the use of skin stapling over conventional ligature because surgical stapling takes less time than conventional suture ligature. Although surgical stapling devices pose various advantages, the conventional surgical stapling devices are expensive and not all clinicians can take advantage of the stapling technology. Also, conventional surgical stapling devices are complex and require skill to operate.

Conventionally, stapling of a wound is accomplished by manually approximating the opposing sides of the wound opening and then positioning the surgical stapler so that a staple will span the opening. The surgical stapler is then manipulated such that the staple is driven into the skin with one leg being driven into each side of the skin and the cross-member of the staple extending across the opening external to the skin surface. Generally, the legs of the staple are driven into an anvil causing the staple to deform to retain the skin tissue in a compressed manner within the staple. The conventional stapler provides an effective manner of closing the wound, however there remains a series of inherent disadvantages in using the conventional stapler. The biggest disadvantage of the conventional stapler is the structure of the staple. The structure of the staple doesn't allow for optimal load transfer across the two sides of the wound, which may result in damaging of the captured tissue. Moreover, the conventional staple doesn't provide optimal and consistent eversion of the wound edges for better cosmetic outcome. Further, the conventional stapler lacks a mechanism for capturing the optimal amount of tissue for the dimension and the structure of the staple, which sometimes capture more or less amount of tissue than the ideal. Capturing more amount of tissue than the required amount leads to pressure necrosis and subsequent scarring. On the other side, capturing less amount of tissue than the ideal may lead to lose closure and subsequent opening and widening of the wound. The other issue with some of the conventional used metal skin staplers is that there is no indicator or a guidance mechanism to guide the surgeon during the application, which may lead to misaligned wound edges. Because of the design of the currently used staple, it poses significant risk of damaging the tissues while entering the same, which may result in “rail-road” scars. There was a need of a stapler that is versatile, and may have different sizes for different applications. Hence, the present disclosure is directed to overcome one or more limitations stated above or any other limitations associated with the conventional systems and methods.

Conventionally, there exits patent literature that discloses about the surgical staplers. For example, the patent application number U.S. Pat. No. 4,662,555A discloses surgical stapler, preferably in a single use disposable form. The surgical stapler includes a combination of a staple feeding mechanism and a staple forming mechanism secured in a frame and an actuating trigger functionally connected to both the staple feeding mechanism and the staple forming mechanism. The staple feeding mechanism includes a rail to guide contained staples serially to a staple forming position and a shuttle in association with the rail as a movable staple retaining cover. The shuttle being functionally connected to the trigger for movement in the same direction and the same time as the staples. This structure provides a reliable surgical stapler in which staples are not likely to jam the staple feeding mechanism.

Further, the patent application number U.S. Pat. No. 5,908,149A discloses a surgical stapler which allows for the multi-directional release of staples. The surgical stapler includes a stapler body and a driver contained therein. A magazine is connected to the stapler body and includes a staple track for carrying a plurality of staples wherein each of the staples has staple legs. An anvil is associated with the staple track for providing a staple forming surface for forming a staple thereon. A feeder element which is spring biased against the staples is used for feeding each staple from the staple track to the anvil. A trigger is operatively connected to the driver and is movable from a pre-fire position to a firing position for advancing the driver against the anvil for forming the staple against the anvil. A kick-off spring is positioned beneath and substantially parallel to the staple track and the anvil. The kick-off spring has a deflectable tip which is deflected away from the anvil and engages the staple legs of the staple by downwardly camming against the staple legs when the trigger is moved from its pre-fire position to its firing position. The kick-off spring ejects the staples off and away from the anvil by disengaging the staple legs with the deflectable tip by upwardly camming against the staple legs upon returning of the trigger from the firing position to the pre-fire position.

The drawbacks/difficulties/disadvantages/limitations of the conventional techniques explained in the background section are just for exemplary purpose and the disclosure would never limit its scope only such limitations. A person skilled in the art would understand that this disclosure and below mentioned description may also solve other problems or overcome the other drawbacks/disadvantages of the conventional arts which are not explicitly captured above.

### SUMMARY OF THE DISCLOSURE

One or more shortcomings of the limitations stated above are overcome by a surgical stapler as claimed, and additional advantages are provided through the provision of the surgical stapler as claimed in the present disclosure. Additional features and advantages are realized through the aspects and techniques of the present disclosure. Other embodiments and aspects of the disclosure are described in detail herein and are considered a part of the claimed disclosure.

In a non-limiting embodiment of the present disclosure, a surgical stapler is disclosed. The surgical stapler includes a stapler body one end of the stapler body being provisioned with a receptacle to receive an affected area of a specimen. A circular base defines a curvilinear path structured to guide one staple of the plurality of staples towards an outlet end defined in the receptacle. The surgical stapler includes a drive mechanism being structured to displace the one staple of the plurality of staples accommodated in the cartridge out of the receptacle and through the affected area received in the receptacle. The drive mechanism includes a conveying member being configured to be displaced between a first position and a second position relative to the curvilinear path. An actuator operatively coupled to the conveying member and being actuatable to displace the conveying member between the first position and the second position relative to the curvilinear path. The conveying member displaces the one staple of the plurality of staples to penetrate the affected area from one end and extend out of the affected area from an other end. The surgical stapler includes a deforming module disposed proximal to an inlet end of the receptacle and structured to deform a portion of the one staple extending out of the affected area from the other end for stapling the one staple relative to the affected area.

In an embodiment of the present disclosure, the stapler body defines an enclosure formed by an elongated base plate. The elongated base plate having a length longer than a width, a sidewall structure extending along a periphery of the elongated base plate about the length. A top cover positioned on the sidewall structure to define a space therebetween.

In an embodiment of the present disclosure, the stapler body defines a bottom opening and a top opening about the width. The bottom opening is structured to receive the receptacle and the top opening structured to receive a cover. The receptacle is integrated with the circular base of the cartridge.

In an embodiment of the present disclosure, the actuator includes a circular disc coupled to the conveying member through a linkage bar. The circular disc being displaceable in at least one of a clockwise direction and an anti-clockwise direction to displace the conveying member by imparting motion through the linkage bar. The conveying member may be at least one of a pusher arm, a conveyor belt and a slider.

In an embodiment of the present disclosure, the actuator includes a plunger extending into the stapler body and operatively engaged with the circular disc. The plunger being displaceable between an extended position and a depressed position to impart rotary movement to the circular disc.

In an embodiment of the present disclosure, the plunger is defined with a plurality of first teeth structured to mesh with a plurality of second teeth defined along a circumference of the circular disc.

In an embodiment of the present disclosure, an end of the plunger out of the stapler body is defined with a handle and an other end of the plunger within the stapler body is defined with a plurality of prongs.

In an embodiment of the present disclosure, the plurality of prongs is structured to displace the deforming module between a resting position and a clamping position during displacement of the plunger to the depressed position. Displacement of the deforming module to the clamping position, deforms the pair of curved arms about the deformation point to staple the one staple relative to the affected area of the specimen.

In an embodiment of the present disclosure, a resilient member disposed between the plunger and the cartridge, wherein the resilient member is configured to bias the plunger towards the extended position from the depressed position.

In an embodiment of the present disclosure, each of the plurality of staples includes a pair of curved arms, an end of the pair of curved arms is defined with a bridge member and the free end of one staple is defined with a tip to penetrate the affected area of the specimen.

In an embodiment of the present disclosure, each arm of the pair of curved arms is defined with a deformation point. A portion of each arm of the pair of curved arms towards the free end being deformable about the deformation point.

In an embodiment of the present disclosure, a plurality of gripping module coupled to the stapler body proximal to the receptacle, the plurality of gripping module configured to grip and draw the affected area into the receptacle area.

In an embodiment of the present disclosure, the receptacle is defined with a guard protruding outwardly from substantially center portion of the receptacle. The guard prevents unintended stapling and injury of structures proximal to the to the affected area of the specimen.

In an another embodiment of the present disclosure, a staple for a surgical stapler. The staple includes a pair of curved arms, one end of the pair of curved arms being connected to each other via a bridge member. Free end of the pair of curved arms defined with a tip to penetrate through the affected area. The pair of curved arms are significantly parallel to each other.

In an embodiment of the present disclosure, the pair of curved arms is defined with a deformation point and a portion of the pair of curved arms towards the tip deforms about the deformation point to form a retaining bridge substantially parallel to the bridge member. The plurality of staples is made of bio-degradable material.

In yet an another embodiment of the present disclosure, a surgical stapler assembly is disclosed. The surgical stapler assembly includes a surgical stapler having a stapler body. One end being provisioned with a receptacle to receive an affected area of a specimen. A plurality of staples, one staple of the plurality of staples defined with a pair of curved arms. One end of the pair of curved arms being connected to each other via a bridge member and free end of the pair of curved arms defined with a tip to penetrate through the affected area.

A cartridge provisioned within the stapler body and being structured to receive and accommodate a plurality of staples. A circular base defines a curvilinear path structured to guide one staple of the plurality of staples towards an outlet end defined in the receptacle. The surgical stapler includes a drive mechanism being structured to displace the one staple of the plurality of staples accommodated in the cartridge out of the receptacle and through the affected area received in the receptacle. The drive mechanism includes a conveying member being configured to be displaced between a first position and a second position relative to the curvilinear path. An actuator operatively coupled to the conveying member and being actuatable to displace the conveying member between the first position and the second position relative to the curvilinear path. The conveying member displace the one staple of the plurality of staples to penetrate the affected area from one end and extend out of the affected area from an other end. The surgical stapler includes a deforming module disposed proximal to an inlet end of the receptacle and structured to deform a portion of the one staple extending out of the affected area from the other end for stapling the one staple relative to the affected area.

It is to be understood that the aspects and embodiments of the disclosure described above may be used in any combination with each other. Several of the aspects and embodiments may be combined together to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF ACCOMPANYING DRAWINGS

The novel features and characteristics of the disclosure are set forth in the description. The disclosure itself, however, as well as a preferred mode of use, further objectives, and advantages thereof, will best be understood by reference to the following description of an illustrative embodiment when read in conjunction with the accompanying drawings. One or more embodiments are now described, by way of example only, with reference to the accompanying drawings wherein like reference numerals represent like elements and in which:

FIG. 1 illustrates an exploded view of a surgical stapler assembly, in accordance with an embodiment of the present disclosure;

FIG. 2a illustrates a side view of the surgical stapler assembly of FIG. 1, in accordance with an embodiment of the present disclosure;

FIG. 2b illustrates an isometric view of the surgical stapler assembly of FIG. 1, in accordance with an embodiment of the present disclosure;

FIG. 2c illustrates a bottom view of a gripping module of the surgical stapler assembly of FIG. 1, in accordance with an embodiment of the present disclosure;

FIG. 2d illustrates an isometric view of a receptacle of the surgical stapler assembly of FIG. 1, in accordance with an embodiment of the present disclosure;

FIG. 2e illustrates an isometric view of a guard of the surgical stapler assembly of FIG. 1, in accordance with an embodiment of the present disclosure;

FIG. 3a illustrates an isometric view of a drive mechanism of the surgical stapler assembly of FIG. 1, in accordance with an embodiment of the present disclosure;

FIG. 3b illustrates an isometric view of a drive mechanism of the surgical stapler assembly of FIG. 1, in accordance with an embodiment of the present disclosure;

FIG. 3c illustrates an isometric view of a plunger of the drive mechanism of FIG. 3a, in accordance with an embodiment of the present disclosure;

FIG. 4a illustrates an isometric view of an actuator of the drive mechanism of FIG. 3a, in accordance with an embodiment of the present disclosure;

FIG. 4b illustrates a side view of an actuator of the drive mechanism of FIG. 3a, in accordance with an embodiment of the present disclosure;

FIG. 5a illustrates a cross sectional view of a cartridge of the surgical stapler assembly of FIG. 1, in accordance with an embodiment of the present disclosure;

FIG. 5b illustrates an isometric view of a deforming module of the surgical stapler assembly of FIG. 1, in accordance with an embodiment of the present disclosure;

FIG. 6a illustrates an isometric view of a staple of the surgical stapler assembly of FIG. 1, in accordance with an embodiment of the present disclosure;

FIG. 6b illustrates an isometric view of the staple of the surgical stapler assembly of FIG. 1, in accordance with an embodiment of the present disclosure;

FIG. 6c illustrates a side view of the staple of the surgical stapler assembly of FIG. 1, in accordance with an embodiment of the present disclosure; and

FIG. 7 illustrates an isometric view of an affected area with a stapler of FIG. 6a, in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION OF THE DISCLOSURE

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that, the conception and specific embodiments disclosed may be readily utilized as a basis for modifying other systems, mechanisms, devices, and assemblies for carrying out the same purposes of the present disclosure. It should also be realized by those skilled in the art that, such equivalent constructions do not depart from the scope of the disclosure as set forth in the appended claims. The novel features which are believed to be characteristics of the disclosure, to its system, together with further objects and advantages will be better understood from the following description when considered in connection with the accompanying figures. It is to be expressly understood, however, that each of the figures is provided for the purpose of illustration and description only and is not intended as a definition of the limits of the present disclosure.

The terms “comprises”, “comprising”, or any other variations thereof, are intended to cover a non-exclusive inclusions, such that a mechanism, an assembly, or a device that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

In accordance with various embodiments of the present disclosure, a surgical stapler is disclosed. The surgical stapler of the present disclosure is convenient and easy to use. The surgical stapler of the present disclosure is simple to operate and is substantially economical compared to conventional surgical stapling devices. The surgical stapler includes a stapler body, one end of the stapler body being provisioned with a receptacle to receive an affected area of a specimen. A cartridge provisioned within the stapler body and being structured to receive and accommodate a plurality of staples. The cartridge being defined with a circular base disposed proximal to the receptacle and an opening on an end opposite to the circular base. The circular base defines a curvilinear path structured to guide one staple of the plurality of staples towards an outlet end defined in the receptacle. The surgical stapler includes a drive mechanism being structured to displace the one staple of the plurality of staples accommodated in the cartridge out of the receptacle and through the affected area received in the receptacle. The drive mechanism may further include a conveying member being configured to be displaced between a first position and a second position relative to the curvilinear path. An actuator operatively coupled to the conveying member and being actuatable to displace the conveying member between the first position and the second position relative to the curvilinear path. The conveying member displace the one staple of the plurality of staples to penetrate the affected area from one end and extend out of the affected area from an other end. The surgical stapler includes a deforming module disposed proximal to an inlet end of the receptacle and structured to deform a portion of the one staple extending out of the affected area from the other end for stapling the one staple relative to the affected area. The surgical stapler of the present disclosure may provide an improved strength and equal force distribution across the wound and prevents the damage of the tissue as compared to the conventional devices.

The following paragraphs describe the present disclosure with reference to FIGS. 1 to 7. In the figure, the same element or elements which have similar functions are indicated by the same reference signs. With general reference to the drawings, a surgical stapler in accordance with the teachings of preferred embodiments of the present disclosure is illustrated and generally identified with reference numeral 100 in the corresponding figures. Other features and elements of the surgical stapler (100) are depicted by respective reference numeral [see list of reference numerals] in the corresponding figures and the same will be used corresponding to respective features henceforth. Accordingly, the drawings are showing only those specific details that are pertinent to understanding the embodiments of the present disclosure so as not to obscure the disclosure with details that will be readily apparent to those of ordinary skill in the art having benefit of the description herein.

The following detailed description is merely exemplary in nature and is not intended to limit application and uses. Furthermore, there is no intention to be bound by any theory presented in the preceding background or summary or the following detailed description. It is to be understood that the disclosure may assume various alternative orientations except where expressly specified to the contrary. It is also to be understood that the specific devices or components illustrated in the attached drawings and described in the following specification are simply exemplary embodiments of the inventive concepts defined in the appended claims. Hence, specific dimensions or other physical characteristics relating to the embodiments that may be disclosed are not to be considered as limiting, unless the claims expressly state otherwise. Hereinafter, preferred embodiments of the present disclosure will be described referring to the accompanying drawings. While some specific terms directed to a specific direction will be used, the purpose of usage of these terms or words is merely to facilitate understanding of the present invention referring to the drawings. Accordingly, it should be noted that the meaning of these terms or words should not improperly limit the technical scope of the present invention.

Further, the term of approximation such as “substantially” when used in reference to physical characteristics in the present disclosure, should be understood to contemplate a range of deviations that would be appreciated by one of ordinary skill in the art to operate satisfactorily for a corresponding use, function, purpose, or the like. The term “substantially” is a descriptive term commonly used in disclosures to avoid a strict numerical boundary to the specified parameter. Any reference to an object in the specification that is preceded by a definite or indefinite article, such as ‘the’, ‘a’, or ‘an’, should be understood to encompass both the singular and the plural forms of the object”. Accordingly, “a” means “at least one/one or more”. The phrase “a/an X” may be construed as “at least one/one or more X”.

Also, it is to be understood that the phraseology and terminology used herein is for description and should not be regarded as limiting. Unless specified or limited otherwise, the terms “connected,” “supported,” “juxtaposed” and “coupled” and variations thereof are used broadly and encompass both direct and indirect mountings, connections, supports, and couplings. Further, “connected” and “coupled” are not restricted to physical or mechanical connections or couplings. It is to be understood that this disclosure is not limited to the specific devices, methods, applications, conditions, or parameters described and/or shown herein and that the terminology used herein is to describe particular embodiments by way of example and is not intended to be limiting of the claimed invention. Hereinafter in the following description, various embodiments will be described. For purposes of explanation, specific configurations and details are outlined to provide a thorough understanding of the embodiments. However, it will also be apparent to one skilled in the art that the embodiments may be practiced without the specific details. Furthermore, well-known features may be omitted or simplified in order not to obscure the embodiment being described. In the forthcoming paragraphs, the surgical stapler (100) is elucidated in detail making reference to FIGS. 1 to 7 in conjunction.

Referring to FIG. 1, illustrates an exploded view of a surgical stapler assembly in accordance with an embodiment of the present disclosure. The surgical stapler assembly (150) among other components may include a surgical stapler (100) and a surgical staple (300) [hereinafter referred to as staple (300)]. The surgical stapler (100) will be elucidated in detail henceforth in conjunction to the figures. The surgical stapler (100) may be configured to staple an affected area (220) of a specimen through a plurality of staples (300). The term “affected area (220) of the specimen” refers to as an area of a living being which is surgically incised or an area which is inflicted with a wound [such as a cut that may be caused accidentally]. However, other type of incisions may also be contemplated herein and may be stapled using the surgical stapler (100) of the present disclosure without deviating from the scope of the present disclosure. The term plurality of staples (300) in the present disclosure refers to the configuration defined here above and below and the plurality of staples (300) of similar configuration being stacked one above the another. The surgical stapler (100) as described may be structured to close the affected area (220) to allow the affected area (220) to heal. In an embodiment, the affected area (220) may be but not limiting to an epithelial tissue, a connective tissue, a muscular tissue and like.

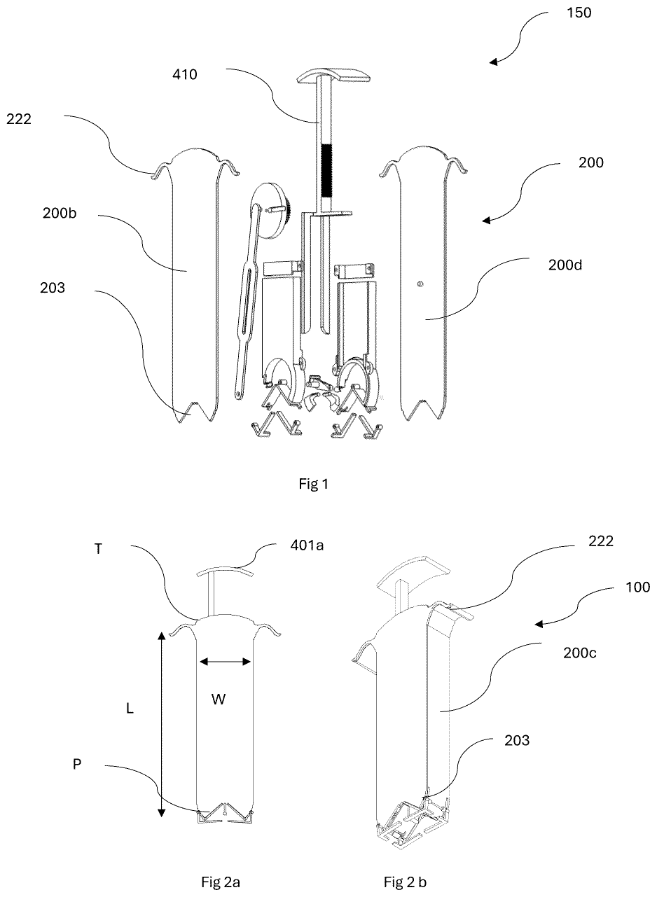

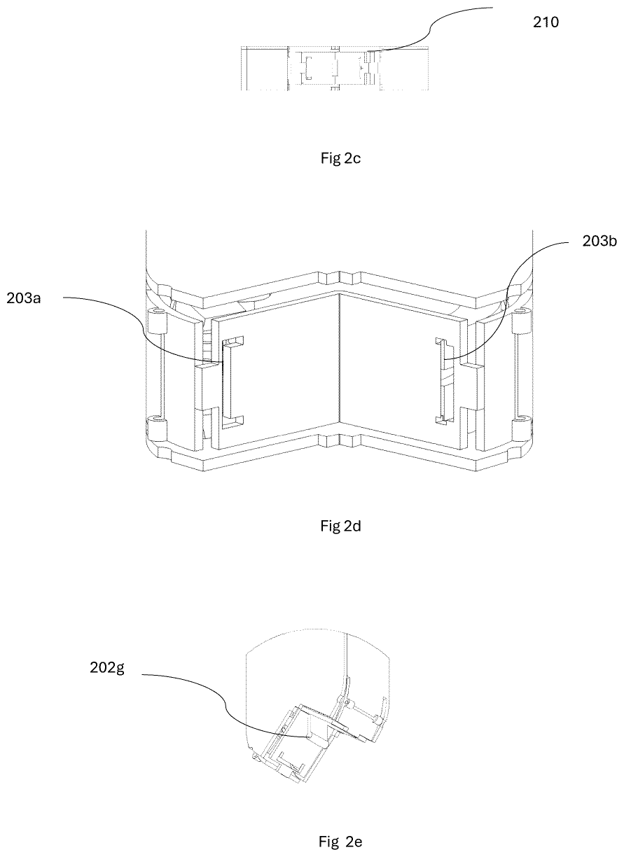

The surgical stapler (100) may have a stapler body (200) that may define an enclosure to receive the other components which will be elucidated in detail in the forthcoming paragraphs. The stapler body (200) may be formed by an elongated base plate (200b) and a top cover complimenting the shape of and spaced apart from the elongated base plate (200b). The elongated base plate (200b) may be defined with a length (L) longer than a width (W). However, the vice-versa i.e., the width (W) being longer than the length (L) may also be contemplated herein without deviating from the scope of the present disclosure. Further, a sidewall structure (200c) may be defined between the elongated base plate (200b) and the top cover (X). Said sidewall structure (200c) may extend in a direction of the length (L) along the periphery of the top cover (X) and the elongated base plate (200b), thereby connecting the same. A space may be defined between the top cover (X) and the elongated base plate (200b) to accommodate the components of the surgical stapler. In an embodiment, the stapler body (200) may include a bottom opening (P) and a top opening (T) defined about the width (W) direction of the stapler body (200). The bottom opening may be structured to receive a receptacle (203), whereas the top opening may be structured to receive a cover (not explicitly illustrated). The shape of the receptacle (203) may be an inverted V-shape, wherein arms of the receptacle (203) may converge into the space defined by the stapler body up to a distance. However, other feasible shapes of the receptacle (203) may be contemplated herein without deviating from the scope of the present disclosure. The receptacle (203) may be structured to receive and hold the affected area (220) of the specimen. As illustrated in FIG. 2d, an inner surface of the receptacle (203) may be defined with an outlet end (203b) and an inlet end (203a). Both the inlet end (203a) and the outlet end (203b) may complement each other such that a plurality of the staples (300) may pass from the outlet end (203b) to the inlet end (203a). In an embodiment, the receptacle (203) may be defined with a plurality of gripping module (210) coupled to the stapler body (200). The plurality of gripping module (210) may be in a ‘L’ shaped structure and may be pivotally connected to the receptacle (203). However, other feasible shapes of the plurality of gripping module (210) may be contemplated herein without deviating from the scope of the present disclosure. The plurality of gripping modules (210) may be configured to grip and draw the affected area (220) into the receptacle (203) such that the affected area (220) is firmly positioned between the outlet end (203b) and the inlet end (203a) to receive the surgical staple (300). The plurality of gripping module (210) ensures that the optimum amount of the affected area is drawn (220) towards the receptacle (203) when the stapler body (200) is positioned on the affected area (220) of the specimen. In an embodiment, the stapler body (200) proximal to the receptacles (203) may be provided with a suction module to draw affected area (220) into the space defined by the receptacle (203). In an alternative embodiment, a plurality of rollers may be provided to draw the affected area (220) into the space defined by the receptacle (203). However, other feasible mechanisms may be employed to draw the affected area (220) without deviating from the scope of the present disclosure.

In an implementation, the receptacle (203) may further be defined with a guard (202g) protruding outwardly from substantially center portion of the receptacle (203). The guard (202g) may be defined with a protuberance at the lower end. The guard (202g) may be structured to prevent unintended stapling and injury of structures proximal to the affected area (220) of the specimen. The guard (202g) may be structured to push the structures proximal to the affected area downwardly during the stapling of the affected area (220). The stapler body (200) may be defined with a gripping member (222) on an end opposite to the end with the receptacles (220). The gripping member (222) may project outwardly and may be substantially perpendicular to the sidewall structure of the stapler body (200). The gripping member (222) may be provided to enable operator to firmly hold the stapler body (200) during stapling operation In an another embodiment, the gripping member (222) may be defined along a portion of the sidewall structure, the top cover and the elongated base plate. For instance, the portion of the gripping member (222) proximal to the top opening may be knurled or may be rubberised to provide suitable grip to the operator during stapling operation. The gripping member (222) may be structured/configured to receive the fingers of operator (i.e., skilled clinician) and provide suitable grip to the operator during the use of the surgical stapler (100).

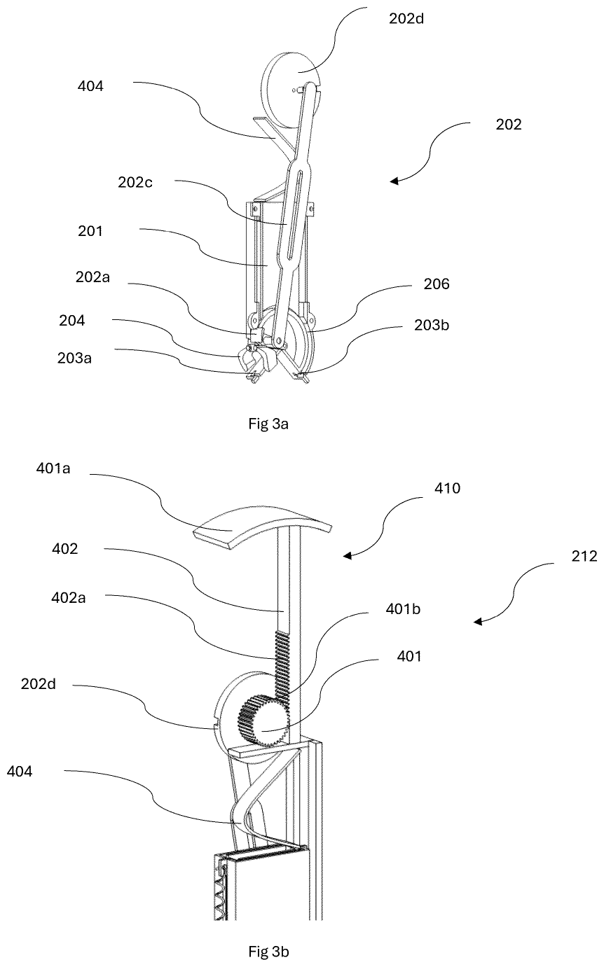

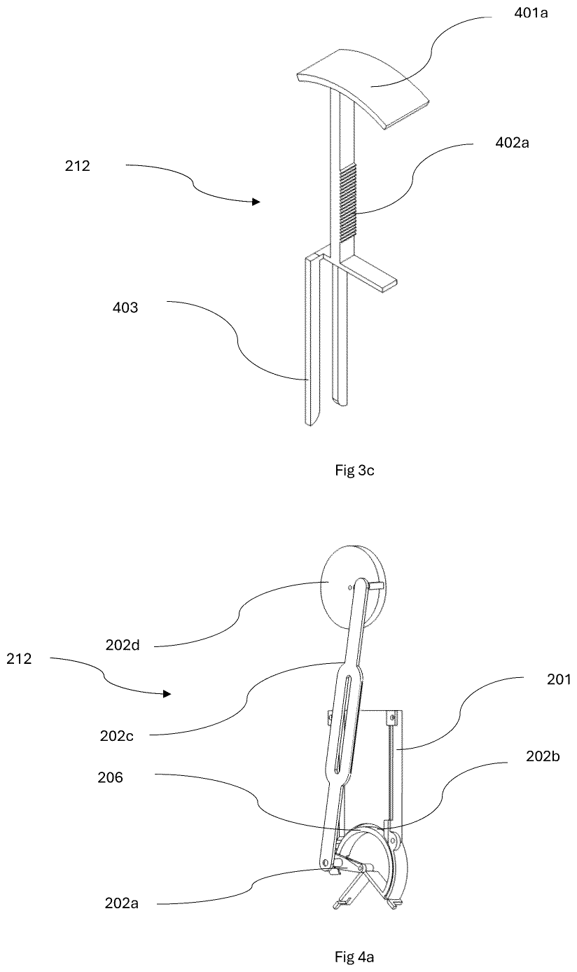

Referring to FIG. 3a and FIG. 3c in conjunction which illustrates a drive mechanism of the surgical stapler. The drive mechanism (202) may include an actuator (212). The actuator (212) among other components that will be elucidate below may include a handle (410). The handle (410) may protrude outwardly from the cover provided on the top opening. A portion of the handle (410) may extend into the stapler body (200) and a remaining portion of the handle (410) may be out of the stapler body (200). The handle (410) may be displaceable between an extended position and a depressed position. In an embodiment, the handle (410) may be actuated manually by the operator or may be operated semi-automatically or automatically without deviating from the scope of the present disclosure. In case of manual operation, the operator may exert pressure on the handle (410) to push the handle (410) in the downward direction [i.e., towards the depressed position] of the surgical stapler (100). In case the handle (410) may be operated semi-automatically or automatically, an actuator may be provisioned. The handle (410) may include a plunger (402) and a head (401a) defined on the plunger (402) to form a gripping means for the operator to displace the plunger (402) between the extended position and the depressed position. Said plunger (402) may partially extend into the space in the stapler body (200) and also a portion of the plunger with the head (401a) resides out of the stapler body. A lower end i.e., an end of the plunger (402) that is within the stapler body (200) and opposite to the head (401a) may be defined with a plurality of prongs (403). In an embodiment, a portion of the plunger (402) may be defined with a plurality of first teeth (402a). The plurality of first teeth (402a) may be structured to mesh with a circular disc (202d) of the actuator (212). The circular disc (202d) may be disposed within the stapler body (200) and may be rotatably connected to either of the top cover or the elongated base plate or both without deviating from the scope of the present disclosure. The circular disc (202d) may be defined with a plurality of second teeth (401b) about a circumferential surface and may be configured to mesh with the plurality of first teeth (402a) on the plunger (402). In an embodiment, a gear wheel (401) may be juxtaposed to one major surface of the circular disc (202d), wherein the gear wheel (401) may be defined with the plurality of second teeth (401b) that may mesh with the plurality of first teeth (402a). The operative engagement between the circular disc (202d) and the plunger (402) may resemble a rack and pinion configuration. However, such configurations should not be construed as a limitation of the present disclosure as feasible configuration may be employed. Displacement of the plunger (402) between the extended position and the depressed position may actuate the rotation of the circular disc (202d) in at least one of a clockwise or an anticlockwise direction. An other major surface of the circular disc (202d) may be operatively coupled to one end of a linkage bar (202c). The linkage bar (202c) may be of any desired shape and size based upon the requirement. Another end opposite to the one end of the linkage bar (202c) may be operatively coupled with a conveying member (202a). The linkage bar (202c) may be configured to displace the conveying member (202a) between a first position and a second position based on the rotational motion of the circular disc (202d) in at least one of the clockwise direction and the anticlockwise direction. The conveying member (202a) may be at least one of a pusher arm, a conveyor belt, a slider arm and the like connected to the linkage bar (202c). The surgical stapler (100) may include a resilient member (404). The resilient member (404) disposed between the plunger (402) and a cartridge (201) [which is elucidated in the forthcoming embodiments] disposed in the stapler body (200). In an embodiment, the cartridge (201) may be defined in the stapler body (200) to accommodate a plurality of staples (300). In an alternate embodiment, the plurality of staples (300) may be accommodated in the stapler body (200), wherein the structure of the stapler body (220) acts as the cartridge (201). The resilient member (404) may be configured to bias the plunger (402) towards the extended position from the depressed position. The resilient member may be at least one of but not limiting to a helical spring, a torsion spring and the like.

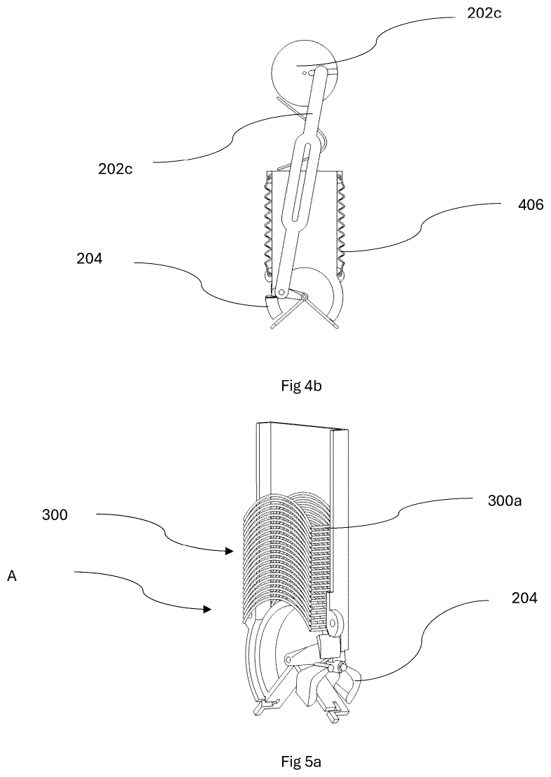

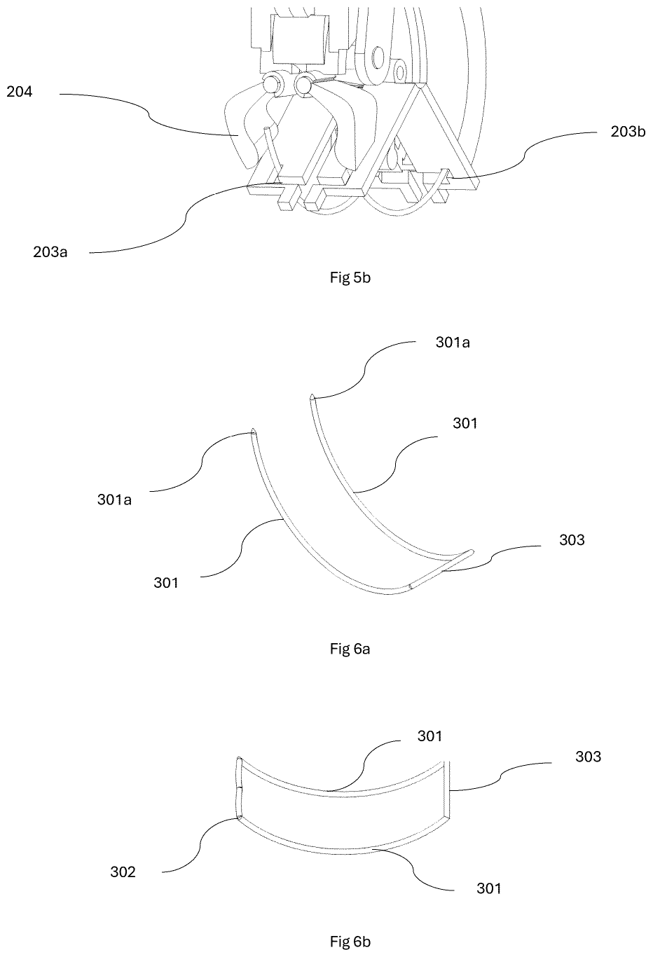

Referring to FIGS. 4a to 5b in conjunction illustrates a driving module in relation with the cartridge (201). As described erstwhile, the surgical stapler (100) may be provided with the cartridge (201) and may be provisioned within the stapler body (200). In the present disclosure, dimensions of the stapler body (200) and the cartridge (201) is not elucidated as the same may be varying based on requirement such as number of staples to be accommodated, dimensions of the staple, type of affected area to be stapled, size of the affected area and the like. The cartridge (201) may be integrally defined with the stapler body (200). In an implementation, the cartridge (201) may be removably disposed in the stapler body (200) without deviating from the scope of the present disclosure. In an embodiment, the cartridge (201) may be structured to receive and accommodate the plurality of staples (300). The cartridge (201) may be defined with an opening at a top end to receive the plurality of staples (300). The cartridge (201) may be provided with a feeding member (406) to retain force and to feed one staple of the plurality of staples (300) on to a base (206) that may be in operational engagement with the cartridge (201). The base (206) may be substantially semicircular or circular and may hereinafter be referred to and interchangeably used as “the circular base”. The circular base (206) may be provisioned proximal to the receptacle at an end opposite to the top end of the cartridge (201). In an embodiment, the circular base (206) may be defined integral to the cartridge (201) at the end opposite to the top end. In an another embodiment, the circular base (206) may be an integral part of the receptacle (203). In an alternate embodiment, the circular base (206) may be disposed in the stapler body (200) relative to and between the receptacle (220) and the cartridge (201) and secured to the stapler body (200). In another implementation, the circular base (206) may be defined integrally with the stapler body (200) without deviating from the scope of the present disclosure. The circular base (206) may be disposed proximal to the receptacle (203) of the stapler body (200). The circular base (206) may be defined with a curvilinear path (202b). In an embodiment, at the center of the circular base (260) the conveying member (202a) may be pivotally coupled. The conveying member (202a) of the drive mechanism (202) may be displaced between the first position (A) and the second position (B) relative to the curvilinear path (202b). The first position (A) [as illustrated in FIG. 5a] is defined as the position in which the one of the staple (300a) of the plurality of staples (300) is on the curvilinear path. The second position (B) [as illustrated in FIG. 7] may be defined as the position in which the one of the staple (300a) of the plurality of staples (300) enters the affected area (220) after the one staple being driven by the conveying member (202a). The curvilinear path (202b) structured to guide the one staple (300a) of the plurality of staples (300) from the outlet end (203b) defined in the receptacle (203) towards the inlet end (203a).

The surgical stapler (100) may include a deforming module (204) disposed proximal to the inlet end (203a) of the receptacle (203). The deforming module (204) may include a first end rotatable about a joint and a second end structured to deform the one staple of the plurality of staples (300) extending into the inlet end (203a). The deforming module (204) may be operable by the plunger (402) through the plurality of prongs (403). The plurality of prongs (403) may displace the deforming module (204) between a resting position and a clamping position. During the clamping position the second end of the deforming module (204) may travel towards each other to deform a portion of the one staple (300a) extending out of the affected area (220) and may staple the one staple (300a) relative to the affected area (220). Such that the portions of the curved arms (301) extending out of the affected area (220) may stay outside the skin surface. In an alternative embodiment, the inlet end (203a) of the receptacle area (203) may be defined with grooves. The grooves may be defined on the surface of the inlet end (203a) and the grooves are configured to deform the portion of the one staple (300a) extending out of the affected area (220) and may staple the one staple (300a) relative to the affected area (220).

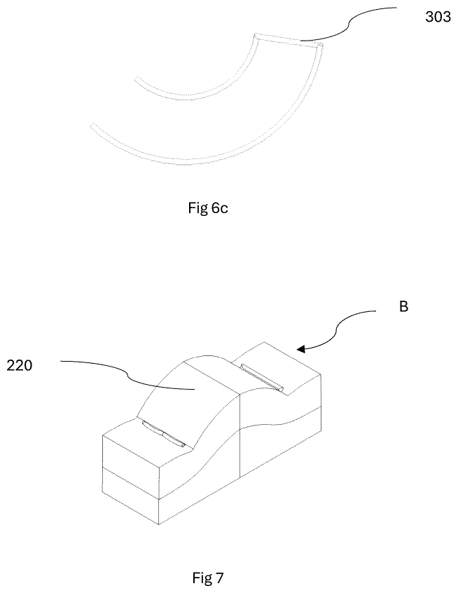

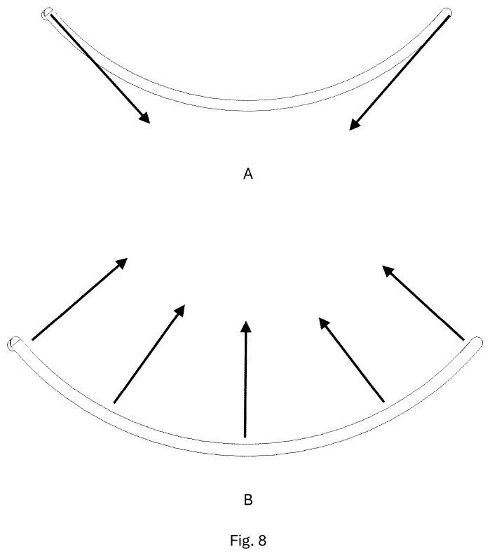

In present embodiment, the plurality of staples (300) are disclosed as illustrated in FIG. 6a to FIG. 7. The plurality of staples (300) may include a pair of curved arms (301). The pair of curved arms (301) may be in substantially parallel plane to each other. The pair of curved arms (301) may have the same arc length and the same curvature. In an another embodiment, one arm of the pair of curved arms (301) may have a different arc length and a different curvature with respect to the another arm of the pair of curved arms (301). This might allow for better shallow wound layer closure along with deep layer wound closure in the required cases. The plurality of curved arms (301) may be provided with one end. The one end of the pair of curved arms (301) being connected to each other via a bridge member (303). The bridge member (303) holds the pair of curved arms (301) from deformation while entering into the affected area. A free end of the pair of curved arms (301) may be defined with a tip (301a) to penetrate through the affected area (220) of the specimen. A portion of the pair of curved arms (301) towards the tip (301a) may be defined with a deformation point (302). Both the pair of curved arms (301) are structured to be deformed inwardly towards each other about the deformation point (302) such that to form a retaining bridge which may or may not be parallel to the bridge member (303). Further, only the bridge member and the portions of the pair of curved arms (301) distal to the deformation point (302) towards the tip may stay outside the skin surface. The plurality of staples (300) may be made of a bio degradable material.

In an operational embodiment, the user aligns the surgical stapler assembly (150) with the affected area. In an embodiment, the affected area (220) may be received in the receptacle (203) through the plurality of gripping module (210). In an alternative embodiment, the affected area (220) may be received in the receptacle upon placing the stapler body (200) about the affected area (220) and slightly inducing the pressure on the stapler body (200) to push the affected area (220) into the receptacle (203). Upon the push of the handle (410), the plunger (402) may be displaced in the depressed position and may impart rotary motion to the circular disc (202d). The circular motion of the circular disc (202d) may be transferred to the conveying member (202a) via the linkage member to displace the conveying member (202a) from the first position (A) to the second position (B) and pushes the one staple (300a) of the plurality of staples (300) out of the outlet end (203b). The one staple (300a) in the second position (B) exits from the outlet end (203b) and penetrates the affected area (220) from one end and enters the inlet end (203a) from another end. Upon further displacement of the handle (410), the plurality of prongs (403) may activate the deforming module (204) that in turn deforms the portion of the pair of curved arms (301) about the deformation point (302). The portion of the pair of curved arms (301) turns inwardly to capture the affected area there between. After the second position (B) the resilient member (404) pushes the plunger (402) into the extended position and the conveying member (202a) may return to it's initial position. The next staple is loaded, and the surgical stapler assembly (150) is ready for the next round.

In the present embodiment, the surgical stapler (100) along with the plurality of staples (300) may provide an improved strength and equal force distribution across the wound and prevents the damage of the tissue as compared to the conventional devices. The surgical stapler assembly (150) is capable of providing bi-layer equivalent closure, which combines the advantages of the deep dermal (removing tension from the skin surface) and the superficial layer closure (wound edge approximation & eversion). The closure can be compared to the combination of the vertical mattress suture and the horizontal mattress suture closures.

### EQUIVALENTS

Embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within scope of the embodiments as described herein.

Throughout this specification the word “comprise”, or variations such as “comprises” or “comprising”, will be understood to imply the inclusion of a stated element, integer or step, or group of elements, integers, or steps, but not the exclusion of any other element, integer or step, or group of elements, integers, or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

### REFERRAL NUMERALS

| Referral numeral | Description |
|---|---|
| Surgical stapler | 100 |
| Surgical stapler assembly | 150 |
| Stapler body | 200 |
| Elongated Base plate | 200b |
| Sidewall structure | 200c |
| Top cover | 200d |
| Cartridge | 201 |
| Drive mechanism | 202 |
| Conveying member | 202a |
| Curvilinear path | 202b |
| Linkage bar | 202c |
| Circular disc | 202d |
| Guard | 202g |
| Receptacle | 203 |
| Deforming module | 204 |
| Circular base | 206 |
| Plurality of Gripping module | 210 |
| Actuator | 212 |
| Affected area | 220 |
| Gripping member | 222 |
| Plurality of staples | 300 |
| One Staple | 300a |
| Plurality of curved arms | 301 |
| tip | 301a |
| Deformation point | 302 |
| Bridge member | 303 |
| Secondary circular disc | 401 |
| Head | 401a |
| Plurality of second teeth | 401b |
| Plunger | 402 |
| Plurality of first teeth | 402a |
| Plurality of Prongs | 403 |
| Resilient member | 404 |
| Feeding member | 406 |
| Handle | 410 |
| First Position | A |
| Second position | B |
| Length | L |
| Bottom opening | P |
| Top opening | T |
| Width | W |

1.-24. (canceled)

25. A surgical stapler (100) comprising:
a stapler body (200), one end of the stapler body (200) being provisioned with a receptacle (203) to receive an affected area (220) of a specimen;
a circular base (206) disposed proximal to the receptacle (203) and, the circular base (206) defines a curvilinear path (202b) structured to guide one staple (300a) of a plurality of staples (300) towards an outlet end (203b) defined in the receptacle (203);
a drive mechanism (202) being structured to displace the one staple (300a) of the plurality of staples (300), the drive mechanism (202) comprises:
a conveying member (202a) being configured to be displaced between a first position (A) and a second position (B) relative to the curvilinear path (202b);
an actuator (212) operatively coupled to the conveying member (202a) and being actuatable to displace the conveying member (202a) between the first position (A) and the second position (B) relative to the curvilinear path (202b) to displace the one staple (300a) of the plurality of staples (300) to penetrate the affected area (220) from one end and extend out of the affected area (220) from an other end; and
a deforming module (204) disposed proximal to an inlet end (203a) of the receptacle (203) and structured to deform a portion of the one staple extending out of the affected area from the other end for stapling the one staple (300a) relative to the affected area (220).

26. The surgical stapler (100) as claimed in claim 25, wherein the stapler body (200) defines a bottom opening and a top opening about the width, the bottom opening structured to receive the receptacle (203) and the top opening structured to receive a cover.

27. The surgical stapler (100) as claimed in claim 25, wherein the circular base (206) is integrated with the receptacle (203).

28. The surgical stapler (100) as claimed in claim 25, wherein the actuator (212) comprises a circular disc (202d) coupled to the conveying member (202a) through a linkage bar (202c), the circular disc (202d) being displaceable in at least one of a clockwise direction and an anti-clockwise direction to displace the conveying member (202a) by imparting motion through the linkage bar (202c).

29. The surgical stapler (100) as claimed in claim 28, wherein the conveying member (202a) may be at least one of a pusher arm, a conveyor belt and a slider.

30. The surgical stapler (100) as claimed in claim 25, wherein the actuator (212) comprises a plunger (402) extending into the stapler body (200) and operatively engaged with the circular disc (202d), the plunger (402) being displaceable between an extended position and a depressed position to impart rotary movement to the circular disc (202d).

31. The surgical stapler (100) as claimed in claim 25, wherein the plunger (402) is defined with a plurality of first teeth structured to mesh with a plurality of second teeth defined along a circumference of the circular disc (202d).

32. The surgical stapler (100) as claimed any claim 25, wherein an end of the plunger (402) out of the stapler body (200) is defined with a head (401a) and an other end of the plunger (402) within the stapler body (200) is defined with a plurality of prongs (403).

33. The surgical stapler (100) as claimed in claim 25, comprises a cartridge (201) provisioned within the stapler body (200) and being structured to receive and accommodate the plurality of staples (300).

34. The surgical stapler (100) as claimed in claim 25, comprises a resilient member (404) disposed between the plunger (402) and the cartridge (201), wherein the resilient member (404) is configured to bias the plunger (402) towards the extended position from the depressed position.

35. The surgical stapler (100) as claimed in claim 25, comprises a plurality of gripping module (210) coupled to the stapler body (200) proximal to the receptacle (203), the plurality of gripping module (210) configured to grip and draw the affected area (220) into the receptacle area.

36. The surgical stapler (100) as claimed in claim 25, wherein the receptacle (203) is defined with a guard (202g) protruding outwardly from substantially center portion of the receptacle (203) wherein the guard (202g) prevents unintended stapling and injury of structures proximal to the affected area of the specimen.

37. The surgical stapler (100) as claimed in claim 25, wherein the plurality of prongs (403) is structured to displace the deforming module (204) between a resting position and a clamping position during displacement of the plunger (402) to the depressed position, displacement of the deforming module (204) from its resting position to the clamping position, deforms the pair of curved arms (301) about the deformation point (302) to staple the one staple relative to the affected area of the specimen.

38. A surgical stapler assembly (150), comprising:
a surgical stapler (100) having a stapler body (200), one end of the stapler body (200) being provisioned with a receptacle (203) to receive an affected area (220) of a specimen;
a plurality of staples (300), one staple (300a) of the plurality of staples (300) defined with a pair of curved arms (301), one end of the pair of curved arms (301) being connected to each other via a bridge member (303) and a free end of the pair of curved arms (301) opposite to the one end defined with the bridge member (303) is defined with a tip (301a) to penetrate through the affected area (220);
a circular base (206) disposed proximal to the receptacle (203) and, the circular base (206) defines a curvilinear path (202b) structured to guide one staple (300a) of a plurality of staples (300) towards an outlet end (203b) defined in the receptacle (203);
a drive mechanism (202) being structured to displace the one staple of the plurality of staples on the curvilinear path defined by the circular base, out of the receptacle and through the affected area received in the receptacle;
a deforming module (204) disposed proximal to an inlet end (203a) of the receptacle (203) and structured to deform a portion of the one staple extending out of the affected area from the other end for stapling the one staple (300a) relative to the affected area (220) and wherein the geometry of the curvilinear path (202b), the curvature of the arms (301), and the position of the deformation points (302) are cooperatively configured such that, upon complete deformation of the staple (300a), only the bridge member (303) and the portions of the curved arms (301) distal to the deformation points (302) towards the tips (301a) remain external to the skin surface of the affected area (220), while the portions of the curved arms (301) proximal to the deformation points (302) towards the bridge member (303) reside within the tissue of the affected area (220).

39. The surgical stapler (100) as claimed in claim 38, wherein each arm of the pair of curved arms (301) is defined with a deformation point (302), a portion of each arm of the pair of curved arms (301) towards the free end being deformable about the deformation point (302).

40. A method of operating a surgical stapler (100) for closing a skin incision or wound of a subject, the surgical stapler (100) comprising a stapler body (200), a receptacle (203) configured to receive an affected tissue region (220), a curvilinear staple path (202b), at least one staple (300) having a pair of curved arms (301) joined by a bridge member (303) and defining a deformation point (302) along each curved arm (301), the method comprising the steps of:
positioning, the receptacle (203) of the stapler (100) such that opposed tissue margins of the affected region (220) are received within the receptacle in an aligned and everted orientation relative to a midline of the stapler;
actuating, a drive mechanism (202) to displace a selected staple (300a) along the curvilinear staple path (202b) from an initial position to an applied position, causing the curved arms (301) of the staple to penetrate the tissue margins along respective curved trajectories;
deforming, distal portions of the curved arms (301) of the staple (300a) at the deformation points (302) by engagement with a deforming module (204), such that the distal portions of the curved arms (301) turn inwardly towards each other to secure the tissue margins together;
releasing the applied staple (300a) from the stapler (100) after deformation, wherein the geometry of the curved arms (301), the position of the deformation points (302), and the curvilinear path (202b) are cooperatively configured such that, upon completion of the stapling step, only the bridge member (303) and those portions of the curved arms (301) distal to the deformation points (302) remain external to the skin surface, while proximal portions of the curved arms (301) reside within dermal tissue, thereby maintaining epidermal eversion and generating an inward closure force with a selectable upward, horizontal, or downward bias relative to the skin surface.
